(19) United States
(12) Patent Application Publication
(10) Pub. No.: US 2026/0265818 A1
(43) Pub. Date: Sep. 10, 2026

(54) STABLE EMULSIONS CONTAINING ARYL-ALKYL SILOXANE FLUIDS

(71) Applicant: Gelest, Inc., Morrisville, PA (US)

(72) Inventors: Kwei-Yu LIU, Lawrenceville, NJ (US); Vladimir PUSHKAREV, Midland, MI (US)

(21) Appl. No.: 19/560,693

(22) Filed: Mar. 9, 2026

Related U.S. Application Data
(60) Provisional application No. 63/769,477, filed on Mar. 10, 2025.

(51) Int. Cl. C12Q 1/686

(52) U.S. Cl. CPC C12Q 1/686

(57) ABSTRACT

A water-in-oil emulsion system with enhanced thermo-mechanical stability, featuring mean particle diameter below about 250 microns and viscosities from 0.5 cSt to 50 cSt is provided. This emulsion system is specifically designed for use in biotech and medical diagnostic microfluidic applications. The emulsions may include a range of aryl-alkyl siloxane fluids and surfactant combinations, demonstrating the superior stability of low-to-medium viscosity emulsions compared to conventional water-polydimethylsiloxane fluids systems.

### CROSS-REFERENCE TO RELATED APPLICATIONS

This application claims priority to U.S. Provisional Application No. 63/769,477, filed Mar. 10, 2025, the disclosure of which is herein incorporated by reference in its entirety.

### BACKGROUND OF THE INVENTION

Aryl-alkyl siloxane fluids are a type of organosilicon compound distinguished by their unique combination of chemical and physical properties, which makes them highly valuable in demanding applications. These siloxane fluids contain aromatic organofunctional groups attached to the silicon atom, imparting performance characteristics that differ significantly from those of conventional polydimethylsiloxane (PDMS) fluids used in aqueous emulsion systems.

Aryl-alkyl siloxane fluids offer notable advantages over conventional PDMS fluids employed in aqueous emulsion applications, including higher density, increased optical density, lower vapor pressure, and reduced formation of cyclic siloxane byproducts. Incorporation of aryl groups increases the molecular weight and density of the fluids, thereby improving emulsion stability and enhancing compatibility with high-density phases. Their elevated refractive index also enhances optical performance, making these materials valuable in optofluidic systems, microscopy imaging, and related optical applications. Furthermore, the reduced volatility of aryl-alkyl siloxanes-stemming from their lower vapor pressure-improves thermal stability and minimizes evaporation losses, supporting uses in higher-temperature or extended-duration processes. In addition, aryl substitutions decrease the propensity for depolymerization, consequently limiting the formation of low-molecular-weight cyclic siloxanes that are associated with environmental and regulatory concerns. Collectively, these attributes position aryl-alkyl siloxane fluids as superior alternatives to PDMS in specialized emulsion applications where enhanced stability, improved optical properties, and reduced byproduct formation are essential.

### RELATED ART

EP 2798089 B1 describes methods and composition for reverse transcriptase-polymerase chain reactions (PCR) applications that are carried out via an emulsion system.

CN 104846112 A discloses a method and composition for emulsion PCR application using various oil phases. In these compositions, low-molecular-weight surfactants are employed to facilitate the emulsion formation, and the use of a co-surfactant system is taught to improve overall performance and reaction characterization. Phenyl trimethicone is used as one or part of the oil phase.

PCT/EP2022/086593 describes a micro-droplet-generating apparatus for PCR applications, in which surfactants such as methyl-cetyl-PEG (20-40%-3-10%-78%-50%) or ABE-3642 are used together with PDMS having a viscosity of 1.5 cSt as the silicone oil phase.

U.S. Pat. No. 6,372,830 describes a water-in-oil emulsion containing a polyorganosiloxane and a polyorganosiloxane-polyoxyalkylene block copolymer with methyl or phenyl grafting groups. The emulsion is taught to remain stable over a wide temperature range, including lower temperatures. The organic groups include aryl substituents such as phenyl and tolyl.

U.S. Pat. No. 10,639,267 discloses a water-in-oil emulsion for cosmetic applications containing pearl particles or aqueous pigments within the aqueous phases. In certain embodiments, emulsifiers such as PEG/PPG-10/1 dimethicone and phenylated siloxanes, including phenyl trimethicone, are used as the oil phase.

U.S. Patent Application Publication No. 2018/0369083 describes a water-in-oil emulsion system for skin-care applications. The internal aqueous phase comprises 60-80% by weight, while the oil phase comprises less than 30%. In this system, phenyl trimethicone and phenyl dimethicone are employed as non-volatile silicone oils.

Despite these developments, there remains a need for an emulsion system for PCR analysis that utilizes aryl-alkyl siloxane fluids capable of providing stability across a broad temperature range, exhibiting reduced formation of low-molecular-weight cyclic siloxanes, and possessing suitable viscosity for use in emulsion-based PCR (ePCR) applications.

### BRIEF SUMMARY OF THE INVENTION

Aspects of the disclosure are directed to a water-in-oil aryl-alkyl siloxane fluid emulsion comprising an aryl-alkyl siloxane fluid continuous phase that includes at least one siloxane fluid of Formula (1), at least one siloxane surfactant, optionally at least one additive, and further comprising an aqueous phase, wherein the emulsion remains stable for at least about 6 hours:

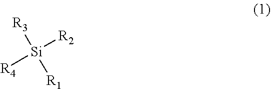

In Formula (1), R₁ is an aryl group, and R₂, R₃, and R₄ are each independently selected from a linear or branched alkyl group having 1 to about 8 carbon atoms, optionally substituted with a siloxy group; a phenyl group, optionally substituted with one or more alkyl groups having 1 to about 10 carbon atoms; an alkyl siloxy group; a dialkyl siloxy group; an arylalkyl siloxy group; or a disiloxy group, provided that at least one of R₂, R₃, and R₄ is a siloxy-containing group.

Advantageous refinements of the invention, which can be implemented alone or in combination, are specified in the dependent claims.

In summary, the following embodiments are proposed as particularly preferred in the scope of the present invention:

Embodiment 1: A water-in-oil aryl-alkyl siloxane fluid emulsion comprising an aryl-alkyl siloxane fluid continuous phase comprising at least one siloxane fluid having Formula (1), at least one siloxane surfactant, optionally at least one additive, and further comprising an aqueous phase, wherein the emulsion remains stable for at least about 6 hours:

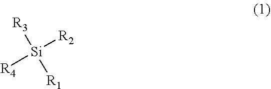

wherein R₁ is an aryl group, R₂, R₃, and R₄ are each independently selected from a linear or branched alkyl group having 1 to about 8 carbon atoms, optionally substituted with a siloxy group; a phenyl group, optionally substituted with one or more alkyl groups having 1 to about 10 carbon atoms; an alkyl siloxy group; a dialkyl siloxy group; an arylalkyl siloxy group; or a disiloxy group, provided that at least one of R₂, R₃, and R₄ is a siloxy-containing group.

Embodiment 2: The emulsion according to Embodiment 1, wherein the surfactant is a grafted siloxane terpolymer containing at least one moiety A having Formula (2), at least one moiety B having Formula (3), at least one moiety C having Formula (4), and a terminal —SiCH₃ group:

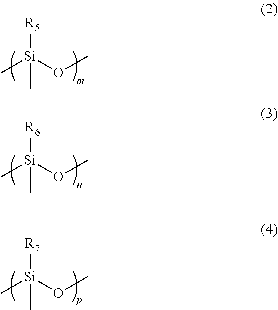

wherein moieties A, B, and C are present in any order; R₅ is methyl; R₆ is a C₃H₆—(CH₂CH₂O)_{q}—OH group; R₇ is a C₁-C₂₀ linear or branched alkyl group or a C₁-C₂₀ phenylalkyl group; q is an integer from about 4 to about 60; m, n, and p are integers from about 1 to about 500; wherein the moiety B content is from 1 mol % to 60 mol %, the moiety C content is from 10 mol % to 50 mol %, and the balance (other than the end group) is moiety A.

Embodiment 3: The emulsion according to Embodiment 2, wherein m, n, and p are each independently integers from about 1 to about 100.

Embodiment 4: The emulsion according to any of Embodiments 1 to 3, wherein R₁ is phenyl, tolyl, dimethylbenzyl, ethylbenzyl, phenylethyl, naphthyl, methylnaphthyl, or biphenyl.

Embodiment 5: The emulsion according to any of Embodiments 1 to 4, wherein the viscosity of the at least one siloxane fluid is less than about 50 cSt at 25° C.

Embodiment 6: The emulsion according to any of Embodiments 1 to 5, wherein the emulsion comprises at least 30 weight % of the at least one siloxane fluid based on a total weight of the emulsion.

Embodiment 7: The emulsion according to Embodiment 6, wherein the emulsion comprises about 30 to about 80 weight % of the at least one siloxane fluid based on the total weight of the emulsion.

Embodiment 8: The emulsion according to any of Embodiments 1 to 7, wherein the emulsion comprises about 9 to about 30 weight % of the at least one siloxane surfactant relative to the aqueous phase of the emulsion.

Embodiment 9: The emulsion according to any of Embodiments 1 to 8, wherein the emulsion comprises about 15 to about 60 weight % saline based on a total weight of the emulsion.

Embodiment 10: The emulsion according to any of Embodiments 1 to 9, wherein a ratio of the aqueous phase to the continuous phase is from about 1.5:8.5 to about 7:3.

Embodiment 11: The emulsion according to any of Embodiments 1 to 10, wherein the emulsion exhibits substantially no phase separation for at least about 6 hours.

Embodiment 12: The emulsion according to any of Embodiments 1 to 11, wherein the emulsion exhibits substantially no measurable change in average particle diameter for at least about 6 hours.

Embodiment 13: The emulsion according to any of Embodiments 1 to 12, wherein the emulsion has a mean particle diameter of below about 250 microns.

Embodiment 14: The emulsion according to Embodiment 13, wherein the emulsion has a mean particle diameter of about 5 microns to about 250 microns.

Embodiment 15: A PCR emulsion comprising the emulsion according to any of Embodiments 1 to 14.

### BRIEF DESCRIPTION OF THE DRAWINGS

The following detailed description of preferred embodiments will be better understood when read in conjunction with the appended drawings. For purposes of illustration, the drawings depict embodiments that are presently considered preferred. It should be understood, however, that the invention is not limited to the precise arrangements or instrumentalities shown.

In the drawings:

FIG. 1 shows photographs demonstrating the performance of surfactants containing different aryl-alkyl siloxane fluids after one hour under UV light;

FIG. 2 shows photographs comparing Emulsion 7 with and without heating cycles;

FIG. 3a is a photograph of Emulsion 7 prior to heat cycles;

FIG. 3b is a photograph of Emulsion 7 after heating cycles;

FIG. 4 illustrates a particle image of an emulsion according to one embodiment of the disclosure; and

FIG. 5 illustrates a particle image of an emulsion according to one embodiment of the disclosure.

### DETAILED DESCRIPTION OF THE INVENTION

Aspects of the disclosure relate to stable emulsions containing aryl-alkyl (such as phenyl-methyl) silicone fluids. These emulsions improve on the performance of conventional dimethylsiloxane fluids components in an emulsion system by substituting some methyl groups with phenyl groups. The emulsions according to the disclosure contain droplets having a mean particle diameter below about 250 microns, a viscosity between about 0.5 cSt and about 50 cSt, and enhanced thermal stability. Without being limited thereto, a desirable application of these emulsions is in PCR for diagnostic devices, which currently employ PDMS- and PFAS-based fluids.

More specifically, the emulsions according to aspects of the disclosure are water-in-oil aryl-alkyl siloxane fluid emulsions containing at least one aryl-alkyl siloxane fluid continuous phase, at least one siloxane surfactant, optionally at least one additive, and an aqueous phase, as described in more detail below. Preferably, such emulsions may be prepared by vigorous agitation until the mixture exhibits a substantially uniform appearance and dispersion. It is understood in the art that the stability of a droplet is related to its resistance to changes over time, including aggregation rate, droplet diameter, shelf life, and pressure and temperature variation. Therefore, the emulsions according to aspects of this disclosure have minimum droplet diameter variation over time, low phase separation, low aggregation, and contain droplets which remain suspended in the solution for at least about 6 hours, preferably at least about 8 hours, such as for about 6 to about 24 hours, or longer.

Unless otherwise stated, any numerical value is to be understood as being modified in all instances by the term “about.” Thus, a numerical value typically includes ±10% of the recited value. For example, the recitation of a temperature such as “10° C.” or “about 10° C.” includes 9° C. and 11° C. and all temperatures there between.

All numerical ranges expressed in this disclosure expressly encompass all possible subranges, all individual numerical values within that range, including integers within such ranges and fractions and decimal amounts of the values unless the context clearly indicates otherwise.

### Siloxane Fluids

The siloxane fluids which are contained in the emulsion are arylated and preferably have general Formula (1):

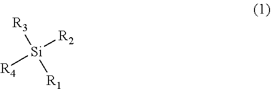

In Formula (1), R₁ is an aryl group such as, but not limited to, phenyl, tolyl, dimethylbenzyl, ethylbenzyl, phenylethyl, naphthyl, methylnaphthyl, or biphenyl. R₂, R₃, and R₄ are each independently selected from a linear or branched alkyl group having 1 to about 8 carbon atoms, optionally substituted with a siloxy group; a phenyl group, optionally substituted with one or more alkyl groups having 1 to about 10 carbon atoms; an alkyl siloxy group; a dialkyl siloxy group; an arylalkyl siloxy group; or a disiloxy group, provided that at least one of R₂, R₃, and R₄ is a siloxy-containing group. For example, and without limitation, R₂, R₃, and R₄ may each independently be selected from methyl, ethyl, propyl, butyl, pentyl, hexyl, heptyl, octyl, phenyl, siloxyalkyl, trimethylsiloxy, ethyldimethylsiloxy, phenyldimethylsiloxy, or dialkyldisiloxy, dimethyldisiloxy, or ethylmethyldisiloxy, but at least one of R₂, R₃, and R₄ must be a siloxy-containing group. Exemplary R₁, R₂, R₃, and R₄ groups are shown below:

### Exemplary R₁ Groups

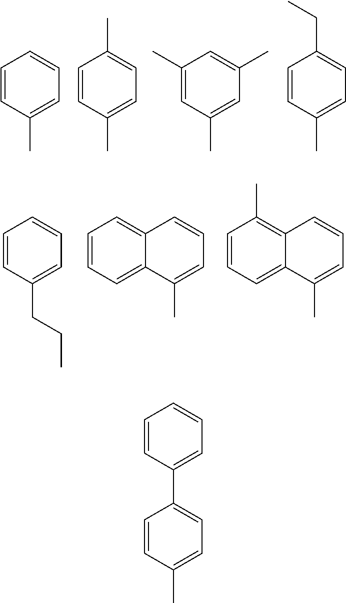

Exemplary R₂, R₃, and R₄ Groups

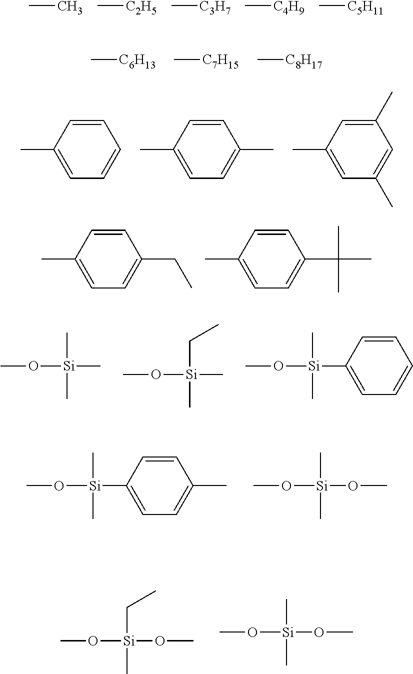

Exemplary preferred compounds having Formula (1) are shown below, in which the substituents are labeled:

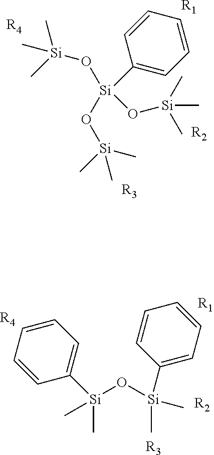

It is within the scope of the disclosure for the emulsion to contain one or more aryl-alkyl siloxane fluids, provided that at least one aryl-alkyl siloxane fluid has Formula (1).

In some preferred embodiments, the continuous phase may include additional components; however, it preferably excludes materials such as mineral oil, which may exhibit lower density, higher vapor pressure, and lower optical density than desired for the formulations described herein. In certain embodiments, the emulsion further excludes particulate or colored components, including magnetic beads, dispersed pigments (e.g., silica, alumina), and coloring agents. In preferred embodiments, the viscosity of the siloxane fluid is below about 50 cSt at 25° C., more preferably from about 0.5 cSt to about 50 cSt. This viscosity range is appropriate for use in PCR analysis, as well as other applications.

### Siloxane Surfactant

In preferred embodiments, the emulsion contains about 9% to about 30 weight % of surfactants by weight relative to the aqueous phase, more preferably about 14 to about 24 weight %, relative to the aqueous phase of the emulsion. Specific surfactants within the scope of the disclosure are described below.

In preferred embodiments, the surfactant is a grafted siloxane terpolymer. For example and without limitation, a presently preferred terpolymer includes three distinct moiety types, designated herein as moieties A, B, and C, which are arranged along the siloxane chain in any order, such that the sequence of A, B, and C units is not limited to a particular pattern and may be random or block in nature. The terpolymer further includes a terminal group of the structure —SiCH₃.

Moiety A has Formula (2), moiety B has Formula (3), and moiety C has Formula (4), as shown below:

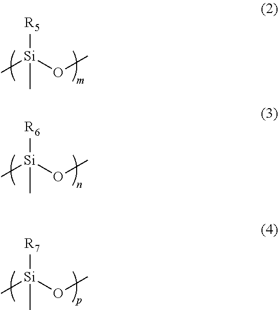

In these formulas, R₅ is methyl; R₆ is a C₃H₆—(CH₂CH₂O)_{q}—OH group; R₇ is a C₁-C₂₀ linear or branched alkyl group or a C₁-C₂₀ phenylalkyl group; and q is an integer representing the number of repeating ethylene oxide units and is from about 4 to about 60.

The relative proportions of the three moieties are controlled to achieve the desired performance. Moiety B is present in an amount from about 1 mol % to about 60 mol %, while moiety C is present in an amount from about 10 mol % to about 50 mol %. The remainder of the terpolymer structure (other than the end group) is composed of moiety A.

The integers m, n, and p independently define the number of repeating units in moieties A, B, and C, respectively, and may be selected to tailor molecular weight, block length, and physical behavior of the terpolymer. The values of m, n, and p may independently range from about 1 to about 500, preferably from about 1 to about 300, and more preferably from about 1 to about 200, most preferably about 1 to about 100.

### Aqueous Phase

In preferred embodiments, the emulsion contains about 15 to about 60 weight % of an aqueous phase, such as about 15 to about 60 weight % deionized water or about 15 to about 60 weight % saline, preferably about 20 to about 55 weight % saline.

### Additives

In certain embodiments directed to emulsion-based PCR applications, the emulsion may optionally comprise one or more additives chosen to enhance droplet stability, dispersion, and integrity during thermal cycling. Suitable optional additives include nonionic wetting and dispersing agents such as Tween-80, which are commonly used in ePCR systems to facilitate uniform droplet formation, minimize coalescence, and maintain stable aqueous compartments throughout the amplification protocol. These additives may be incorporated individually or in combination to support consistent droplet size distribution, preserve compartmentalization of nucleic-acid templates, and ensure reliable downstream amplification performance.

### General Considerations

It is within the scope of the disclosure to include additional components in the emulsions described herein, such as, but not limited to, additional organic water-soluble surfactants, inorganic salts, or oil-phase additives such as octyl trimethoxysilane or amino-functionalized siloxanes.

Preferred emulsions according to aspects of the disclosure have an aqueous phase to continuous phase ratio of about 1.5:8.5 to about 7:3, preferably about 2:8 to about 6:4.

Emulsions according to the disclosure preferably contain droplets having a mean particle diameter of about 5 microns to about 250 microns, preferably about 20 microns to about 150 microns. In a preferred method, the mean particle diameter is determined by analyzing a microscopic photo and measuring the diameter of 50 droplets. However, the population of droplets may be greater than 50 and some tiny droplets may be difficult to measure edge to edge.

In preferred embodiments, the emulsion contains at least about 30 weight % continuous phase (siloxane fluid), preferably about 35 to about 85 weight %, more preferably about 40 to about 80 weight %, even more preferably about 55 to about 77 weight % aryl-alkyl siloxane fluid(s). That is, it is within the scope of the disclosure for the emulsion to contain about 30 to about 80 weight % of at least one aryl-alkyl siloxane fluid based on the total weight of the emulsion.

### Stability and Applications

The emulsions described herein have desirable stability for prolonged periods upon heating to elevated temperatures. For example, the exemplary emulsions described in FIG. 2 have been found to be stable for at least 6 hours at room temperature, or 2 hours after repeated heating cycles every 3 minutes for 2 hours between 20° C. and 99° C. The detailed results of this stability test will be discussed in the following sections.

As used herein, “stable” refers to an emulsion that exhibits substantially no phase separation and substantially no measurable change in average particle diameter over the relevant period, including for at least about 6 hours under the conditions described.

For the purpose of this disclosure, the phrase “substantially no phase separation” refers to the absence of any meaningful or functionally significant separation of the aqueous and continuous phases under the conditions of evaluation. Minor or superficial visual changes, such as slight creaming or the appearance of a faint interfacial layer, particularly but not only when observed only under extreme conditions (e.g., near the upper end of the test temperature range, such as around 100° C.), are considered insufficient to constitute phase separation for purposes of this definition, provided that the emulsion remains physically integrated and continues to exhibit its intended functional performance. That is, an emulsion which exhibits these or other minor or superficial visual changes may still meet the definition of “stable” for the purposes of this disclosure.

For the purpose of this disclosure, the phrase “substantially no measurable change in average particle diameter” refers to changes in particle size that are sufficiently small that they do not materially affect the stability, performance, or functional characteristics of the emulsion. Such changes may include minor variations arising from normal measurement uncertainty, thermal expansion effects, or expected microscopic restructuring of the aqueous phase. In certain embodiments, a change in the average particle diameter of up to about 10-20 μm over the evaluated period is considered to constitute substantially no measurable change.

While not limited thereto, the emulsions according to the disclosure are appropriate for use in PCR applications, due to their favorable viscosity, particle size (mean particle diameter), and temperature stability properties. Accordingly, aspects of the disclosure also relate to PCR emulsions containing the components described above.

The invention will now be described in conjunction with the following, non-limiting examples.

### Examples

The following standard analytical equipment and methods are used in the examples:

Image microscope: a Teledyne Infinity microscope was used for particle image capturing. 20 microliter of sample was withdrawn from the emulsion and added into the coverslip on the microscope slide, which contained a 10×10 grid with size 0.3 millimeter. Images were taken right after preparation, after 4 hours, and after 8 hours to examine the condition of the emulsion particles

Stability Test: An emulsion was prepared by mixing a surfactant, siloxane fluids, and aqueous phase in an Eppendorf tube with a total weight of approx. 1.48 g, which included the combination of saline, surfactants and siloxane fluids. The Eppendorf tube was rested on a stand and the emulsion allowed to settle over time, with pictures taken at specific time intervals to record the stability of the emulsion.

Saline solution was prepared by dissolving NaCl in deionized water (100-500 mM). In addition, fluorescein was added into water at a 2.5 g/1 L concentration for visual differentiation between aqueous and oil phases: an emulsion containing saline illuminates a yellow-green color under UV.

Surfactants. Two surfactants D and E were employed in the described Examples:

Surfactant D comprises (28% dodecylmethylsiloxane)-[4% hydroxy(polyethyleneoxy(6-9)propyl)methylsiloxane]-(68% dimethylsiloxane) terpolymer

Surfactant E comprises (36% dodecylmethylsiloxane)-[14% hydroxy(polyethyleneoxy(4-7)propyl)methylsiloxane]-(50% dimethylsiloxane) terpolymer.

### Effect of Siloxane Fluids Structure on Emulsion Stability

Preparation of Emulsions Aryl-alkyl fluids for emulsions classified into two types/classes of siloxane fluids, phenylated and phenyl-free, were studied, as described in Emulsions 1 to 6 below. Phenyl trimethicone, 1,3-diphenyltetramethyldisiloxane, and a homopolymer of phenylmethyl siloxane were employed as representative phenylated siloxanes. Hexamethyldisiloxane, methoxy-terminated poly(methylsilsesquioxane), and polydimethylsiloxane were employed as representative phenyl free siloxanes.

Each of the following emulsions contained Surfactant D, a siloxane fluid, and 200 mM saline as the aqueous phase. The surfactant, saline, and siloxane fluids were mixed for 30 seconds by vortex to form each emulsion.

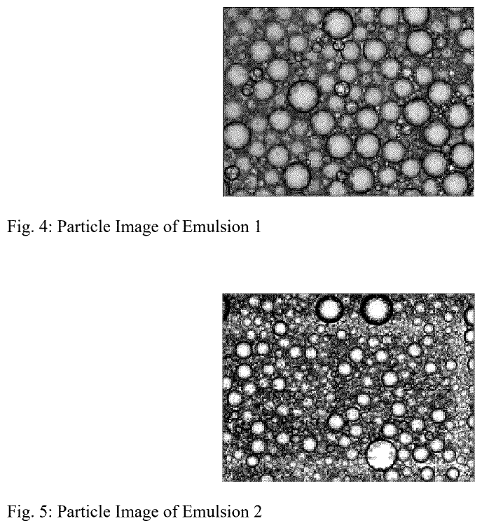

Emulsion 1: Surfactant D (0.084 g, 20% by weight of the aqueous phase) was added into an Eppendorf tube, followed by phenyl trimethicone (0.977 g) and saline solution (0.419 g). The tube was inverted and hand-shaken to allow all components to gently mix, and then the mixture solution was mixed by vortex for 30 seconds. The emulsion remained yellow green under UV for at least 6 hours, which indicated that the emulsion was stable. The particle diameters were 18-74 microns, as shown in FIG. 4.

Emulsion 2: Surfactant D (0.084 g, 20% by weight of the aqueous phase) was added into an Eppendorf tube, followed by 1,3-diphenyltetramethyldisiloxane (0.977 g) and saline solution (0.419 g). The tube was inverted and hand-shaken to allow all components to gently mix, and then the mixture solution was mixed by vortex for 30 seconds. The solution remained yellow green under UV for at least 24 hours, which indicates that the emulsion was stable. The particle diameters were 24-45 microns, as shown in FIG. 5.

Emulsion 3: Surfactant D (0.084 g, 20% by weight of the aqueous phase) was added into an Eppendorf tube, followed by hexamethyldisiloxane (0.977 g) and saline solution (0.419 g). The emulsion separated into two phases immediately.

Emulsion 4: Surfactant D (0.084 g, 20% by weight of the aqueous phase) was added into an Eppendorf tube, followed by polydimethylsiloxane (0.977 g) and saline solution (0.419 g). The viscosity of the polydimethylsiloxane ranges from 5-10 cSt. The tube was inverted and hand-shaken to allow all components to gently mix, and then the mixture solution was mixed by vortex for 30 seconds. The emulsion aggregated.

Emulsion 5: Surfactant D (0.084 g, 20% by weight of the aqueous phase) was added into an Eppendorf tube, followed by methoxy-terminated poly(methylsilsesquioxane) (0.977 g) and saline solution (0.419 g). The tube was inverted and hand-shaken to allow all components to gently mix, and then the mixture solution was mixed by vortex for 30 seconds. The emulsion aggregated.

Emulsion 6: Surfactant D (0.084 g, 20% by weight of the aqueous phase) was added into an Eppendorf tube, followed by phenylmethylsiloxane homopolymer (0.977 g) and saline solution (0.419 g). The tube was inverted and hand-shaken to allow all components to gently mix, and then the mixture solution was mixed by vortex for 30 seconds. The particle diameters were 20 to 44 microns, and the emulsion was stable for at least 6 hours.

Emulsion 7: Surfactant D (0.13 g, 20% by weight of the aqueous phase) was added into an Eppendorf tube, followed by 1,3-diphenyltetramethyldisiloxane (0.673 g) and saline solution (0.673 g). The tube was inverted and hand-shaken to allow all components to gently mix, and then the mixture solution was mixed by vortex for 30 seconds. The solution remained yellow green under UV for at least 24 hours, which indicates that the emulsion was stable. The particle diameters were 21-35 microns.

### Analysis of Emulsions Prepared Using Surfactant D

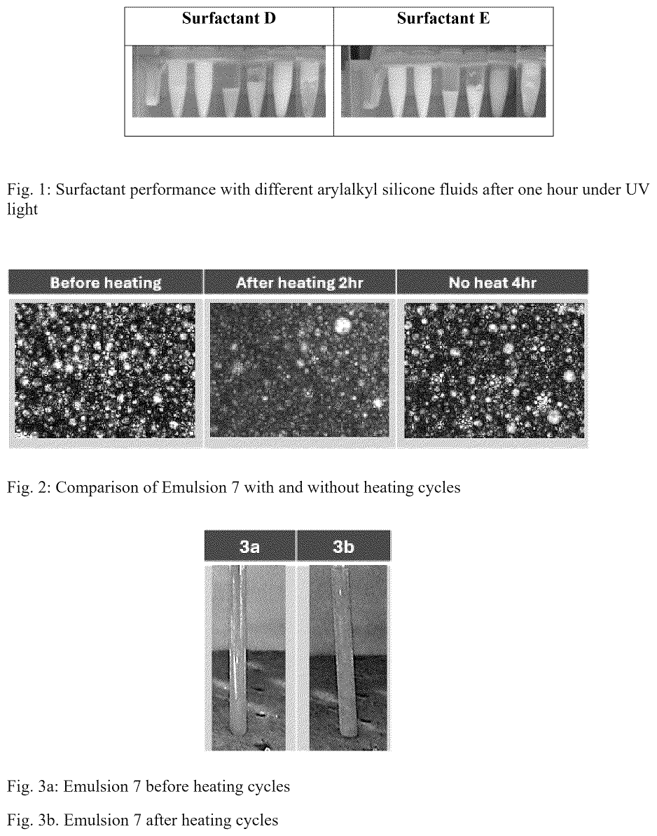

It was found that the emulsions containing phenylated siloxane fluids, such as phenyl trimethicone and 1,3-diphenyltetramethyldisiloxane, were relatively stable, remaining as a single phase for at least 6 hours. In contrast, the emulsions prepared with phenyl free siloxanes, such as hexamethyldisiloxane and polydimethylsiloxane, were unable to form stable emulsion, as shown in FIG. 1.

This behavior is attributed to the solubility of the respective siloxane toward Surfactant D: Surfactant D was soluble in phenylated siloxanes, but insoluble in phenyl-free siloxanes. The presence of nonpolar phenyl groups also facilitated the solvation of Surfactant D. Phenyl-free siloxane fluids, such as polydimethylsiloxane and hexamethyldisiloxane, demonstrated significant aggregation in emulsion with Surfactant D. In contrast, phenylmethyl siloxane homopolymer was able to generate emulsion droplets.

Thus, emulsion solutions prepared with phenylated siloxane fluids were stable, with particle diameter showing an inverse relationship to the number of phenyl substituents present in the structure. The number of phenyl groups also showed proportionality to the particle's suspension time. Emulsions prepared using phenyl trimethicone (one phenyl group) had mean particle diameters of 18 to 75 microns lasting for over six hours, and emulsions containing 1,3-diphenyltetramethyldisiloxane (two phenyl groups) had average particle diameter ranging from 24 to 45 microns, yet the stability of the emulsion lasted more than 24 hours. Without being bound by theory, this may be explained by the hydrophobicity of arylalkyl siloxane fluids and the interaction between phenyl groups to stabilize the emulsion. This phenomenon was confirmed by an emulsion system containing 1,3-diphenyltetramethyldisiloxane and Surfactant D. The aromatic interaction was significantly higher, and it resulted in higher viscosity, smaller particle diameter, and prolonged emulsion suspension in the solution.

The properties of these emulsions are summarized in Table 1.

TABLE 1
Emulsion particle diameters and settling time
with different siloxane oils/surfactant

| | Surfactant | |
|---|---|---|
| Siloxane fluid | Surfactant D | Surfactant E |
| Phenyl trimethicone | 18-74 micron, 6 hr | 17-28 micron, 6+ hr |
| 1,3-diphenyltetramethyl- | 24-44 micron, 24+ hr | 8-26 micron, 6+ hr |
| disiloxane | | |
| Hexamethyldisiloxane | Phase Separates | Phase Separates |
| Polydimethylsiloxane | Aggregates | Aggregates |
| Phenylmethyl siloxane | 20-44. micron, 6+ hr | 14-29 micron, 6+ hr |
| homopolymer | | |
| Methoxy-terminated | Aggregates | Aggregates |
| poly(methylsilsesquioxane) | | |

The performance of the bulk emulsions from each siloxane fluid are shown in FIG. 1. Specifically, FIG. 1 includes photographs which show the surfactant performance of the different arylalkyl silicone fluids after one hour (under UV light) for both Surfactants D and E. From left to right, each set includes phenyl trimethicone, 1,3-diphenyltetramethyldisiloxane, hexamethyldisiloxane, polydimethylsiloxane, homopolymer of phenylmethyl siloxane, and methoxy-terminated poly(methylsilsesquioxane).

### Study of Emulsions Prepared Using Surfactants E

Preparation of Emulsions: Emulsions were prepared using Surfactant E and the following fluids: phenyl trimethicone, 1,3-diphenyltetramethyldisiloxane, and homopolymer of phenylmethyl siloxane as phenylated fluids, and hexamethyldisiloxane, methoxy-terminated poly(methylsilsesquioxane) and polydimethylsiloxane as phenyl free fluids.

Each of the following emulsions contained Surfactant E, a siloxane fluid, and 200 mM saline as the aqueous phase. The surfactant, saline, and siloxane fluids were mixed for 30 seconds by vortex to form each emulsion.

Emulsion 8: Surfactant E (0.084 g, 20% by weight of the aqueous phase) was added into an Eppendorf tube, followed by phenyl trimethicone (0.977 g) and saline solution (0.419 g). The tube was inverted and hand-shaken to allow all components to gently mix, and then the mixture solution was mixed by vortex for 30 seconds. The emulsion remained yellow green under UV for at least 6 hours. The diameters of emulsion particles were 17 to 28 microns.

Emulsion 9: 0.084 g Surfactant E (20% by weight of the aqueous phase) was added into an Eppendorf tube, then 1,3-diphenyltetramethyldisiloxane (0.977 g) and saline solution (0.419 g) were added into the Eppendorf tube. The tube was inverted and hand-shaken to allow all components to gently mix, and then the mixture solution was mixed by vortex for 30 seconds. The emulsion remained yellow green for at least 6 hours, which indicates that the emulsion was stable. The diameters of emulsion particles were 8 to 26 microns.

Emulsion 10: Surfactant E (0.084 g, 20% by weight of the aqueous phase) was added into an Eppendorf tube, followed by hexamethyldisiloxane (0.977 g) and saline solution (0.419 g). The emulsion separated into two phases immediately.

Emulsion 11: Surfactant E (0.084 g, 20% by weight of the aqueous phase) was added into an Eppendorf tube, followed by polydimethylsiloxane (0.977 g) and saline solution (0.419 g). The viscosity of polydimethylsiloxane ranges from 5-10 cSt. The tube was inverted and hand-shaken to allow all components to gently mix, and then the mixture solution was mixed by vortex for 30 seconds. The emulsion aggregated.

Emulsion 12: Surfactant E (0.084 g, 20% by weight of the aqueous phase) was added into an Eppendorf tube, followed by methoxy-terminated poly(methylsilsesquioxane) (0.977 g) and saline solution (0.419 g). The tube was inverted and hand-shaken to allow all components to gently mix, and then the mixture solution was mixed by vortex for 30 seconds. The emulsion aggregated.

Emulsion 13: Surfactant E (0.084 g, 20% by weight of the aqueous phase) was added into an Eppendorf tube, followed by phenylmethylsiloxane homopolymer (0.977 g) and saline solution (0.419 g). The tube was inverted and hand-shaken to allow all components to gently mix, and then the mixture solution was mixed by vortex for 30 seconds. The emulsion remained yellow green under UV for at least 6 hours. The particle diameters of the emulsion were 14 to 29 microns.

The results of the analysis of these emulsions are shown in Table 1. It was observed that surfactant E generated smaller droplets than Surfactant D. Similar to Surfactant D, emulsions that were prepared with polydimethylsiloxane remained unstable and phase separated; however, emulsions were successfully prepared with phenylated siloxane fluids. The numbers of particles were notably smaller compared to emulsion made with surfactant D.

### Temperature Stability Test

Emulsion 7, as described above, was added to different tubes. The emulsions were subjected to heating cycles: between 20° C. and 99° C. every 3 minutes for 2 hours (40 cycles in total).

As shown in FIG. 2, Emulsion 7 remained stable through the entire heating cycle. After completion of the heat-cycling study, Emulsion 7 exhibited only modest changes in their average particle diameters. Specifically, Emulsion 7 showed an decrease in average particle diameter from about 28 μm to about 26 μm. The control sample, which was maintained at ambient temperature without heating, also remained stable. The control emulsions exhibited only a slight increase of about 1 μm even after 4 hr. These results demonstrate that the emulsions maintained their structural integrity throughout the study, with only minor variations in droplet size consistent with normal thermal or measurement-related effects. The observed changes are illustrated in FIGS. 3a and 3b, where FIG. 3a presents Emulsion 7 (left) prior to heat cycling, and FIG. 3b presents Emulsion 7 after completing the heating cycles.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A water-in-oil aryl-alkyl siloxane fluid emulsion comprising an aryl-alkyl siloxane fluid continuous phase comprising at least one siloxane fluid having Formula (1), at least one siloxane surfactant, optionally at least one additive, and further comprising an aqueous phase, wherein the emulsion remains stable for at least about 6 hours: 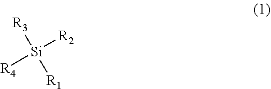
wherein R₁ is an aryl group, R₂, R₃, and R₄ are each independently selected from a linear or branched alkyl group having 1 to about 8 carbon atoms, optionally substituted with a siloxy group; a phenyl group, optionally substituted with one or more alkyl groups having 1 to about 10 carbon atoms; an alkyl siloxy group; a dialkyl siloxy group; an arylalkyl siloxy group; or a disiloxy group, provided that at least one of R₂, R₃, and R₄ is a siloxy-containing group.

2. The emulsion according to claim 1, wherein the surfactant is a grafted siloxane terpolymer containing at least one moiety A having Formula (2), at least one moiety B having Formula (3), at least one moiety C having Formula (4), and a terminal —SiCH₃ group: 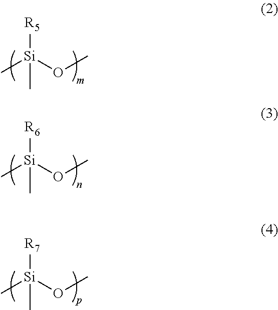
wherein moieties A, B, and C are present in any order; R₅ is methyl; R₆ is a C₃H₆—(CH₂CH₂O)_{q}—OH group; R₇ is a C₁-C₂₀ linear or branched alkyl group or a C₁-C₂₀ phenylalkyl group; q is an integer from about 4 to about 60; m, n, and p are integers from about 1 to about 500; wherein the moiety B content is from 1 mol % to 60 mol %, the moiety C content is from 10 mol % to 50 mol %, and the balance (other than the end group) is moiety A.

3. The emulsion according to claim 2, wherein m, n, and p are each independently integers from about 1 to about 100.

4. The emulsion according to claim 1, wherein R₁ is phenyl, tolyl, dimethylbenzyl, ethylbenzyl, phenylethyl, naphthyl, methylnaphthyl, or biphenyl.

5. The emulsion according to claim 1, wherein the viscosity of the at least one siloxane fluid is less than about 50 cSt at 25° C.

6. The emulsion according to claim 1, wherein the emulsion comprises at least 30 weight % of the at least one siloxane fluid based on a total weight of the emulsion.

7. The emulsion according to claim 6, wherein the emulsion comprises about 30 to about 80 weight % of the at least one siloxane fluid based on the total weight of the emulsion.

8. The emulsion according to claim 1, wherein the emulsion comprises about 9 to about 30 weight % of the at least one siloxane surfactant relative to the aqueous phase of the emulsion.

9. The emulsion according to claim 1, wherein the emulsion comprises about 15 to about 60 weight % saline based on a total weight of the emulsion.

10. The emulsion according to claim 1, wherein a ratio of the aqueous phase to the continuous phase is from about 1.5:8.5 to about 7:3.

11. The emulsion according to claim 1, wherein the emulsion exhibits substantially no phase separation for at least about 6 hours.

12. The emulsion according to claim 1, wherein the emulsion exhibits substantially no measurable change in average particle diameter for at least about 6 hours.

13. The emulsion according to claim 1, wherein the emulsion has a mean particle diameter of below about 250 microns.

14. The emulsion according to claim 13, wherein the emulsion has a mean particle diameter of about 5 microns to about 250 microns.

15. A PCR emulsion comprising the emulsion according to claim 1.
